(19) United States
(12) Patent Application Publication
(10) Pub. No.: US 2011/0021595 A1
(43) Pub. Date: Jan. 27, 2011

(54) KETOROLAC TROMETHAMINE COMPOSITIONS FOR TREATING OR PREVENTING OCULAR PAIN

(75) Inventors: Eldon Q. Farnes, Laguna Beach, CA (US); Mayssa Attar, Placentia, CA (US); Rhett M. Shiffman, Laguna Beach, CA (US); Chin-Ming Chang, Tustin, CA (US); Richard S. Graham, Irvine, CA (US); Devin F. Welty, Foothill Ranch, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 12/685,074

(22) Filed: Jan. 11, 2010

(30) Foreign Application Priority Data
Jul. 23, 2009 (AU) 2009202969

(51) Int. Cl. A61K 31/407; A61P 27/02

(52) U.S. Cl. 514/413

(57) ABSTRACT

The present invention provides an aqueous ophthalmic solution comprising an effective amount of ketorolac which comprises mixtures of carboxymethyl cellulose in an aqueous solution wherein said concentration of carboxymethyl cellulose is selected to provide an increased absorption of ketorolac in the eye and improve visual acuity of the users.

### RELATED APPLICATION

This application claims the benefit under 35 USC 119 of Australian Patent Application Serial No. 2009202969, filed Jul. 23, 2009, the disclosure of which is hereby incorporated in its entirety herein by reference.

### FIELD OF THE INVENTION

This invention relates to topical ophthalmic solutions comprising mixtures of carboxymethyl cellulose and ketorolac for treating and preventing post-operative ocular pain wherein the ophthalmic solutions of the present invention has increased bioavailability of ketorolac to the cornea and improves visual acuity of the user.

### DESCRIPTION OF THE RELATED ART

The present invention relates to compositions and methods useful for administering an ophthalmic therapeutic component to a human or animal. More particularly, the present invention relates to compositions and methods which are very effective in facilitating administration of medication through a cornea of an eye and improving the vision of the patient.

Topical nonsteroidal anti-inflammatory drugs (NSAIDs) are used to control pain and postoperative inflammation. All drugs are associated with some adverse effects. With the use of NSAIDS in topical ophthalmic treatment of patients, surface toxicity has been a concern, and incidents of keratitis, corneal subepithelial infiltrates, ulceration, and corneal melts have been reported (Guidera et al, Opthalmology, 2001, 108 (5), pp. 936-944; Solomon et al, J Cataract Refract Surg, 2001, 27 (8), pp. 1232-1237; Teal et al, J Cataract Refract Surg, 1995, 21(5), pp. 516-518). Further, patients often report burning or stinging on instillation (Jaanus et al, Antiinflammatory Drugs. Clinical Ocular Pharmacology, Bartlet, J. D. and Jaanus, S. D., Ed., Boston: Heineman, 2001, pp. 265-298). The burning or stinging could be related to the concentration of the active component of the formulation.

Ketorolac tromethamine 0.5% (w/v) ophthalmic solution, available from Allergan, Inc. and marketed under the trade name ACULAR®, is a safe and effective NSAID with proven analgesic and anti-inflammatory activity. The most common adverse event associated with the use of the 0.5% ketorolac formulation is ocular irritation, primarily burning and stinging upon instillation. Ketorolac tromethamine 0.4% (w/v) ophthalmic solution, also available from Allergan, Inc. and marketed under the trade name ACULAR LS®, has shown improved bioavailability and less stinging on instillation than ACULAR®, but there remains a need for an improved ketorolac tromethamine formulation with significantly improved bioavailability and greater tolerability to improve patient compliance, minimized ocular surface toxicity, improved patient comfort, increased retention time of the active ingredient and improved wound healing capabilities during use.

It is an object of this invention to provide a ketorolac formulation for instillation in the eye to eliminate or reduce ocular irritation, to prevent inflammation due to allergic conjunctivitis, to improve tolerability, patient compliance, duration and effect of ketorolac, to allow for reduction of dosing from four times daily to twice daily, and to increase the effectiveness of treatment by being free of benzalkonium chloride or other preservatives which may cause discomfort.

It is another object of the invention to significantly improve bioavailability and to increase the ocular absorption of ketorolac yet provide an aqueous solution having an optimized concentration of ketorolac and improving the patients visual acuity during use of the product.

It is another object of the invention to extend the effects of ketorolac and allow for a decrease in required daily dosage by up to 50%.

It is another object of the invention to provide reduction of inflammation associated with cataract surgery and reduction of pain associated with cataract surgery in comparison to other ketorolac formulations.

It is another object of the invention to create a ketorolac formulation with improved wound healing capabilities.

Other objects of this invention will become apparent from a reading of the present specification.

### SUMMARY OF THE INVENTION

The present invention provides an aqueous ophthalmic formulation comprising an effective and optimized amount of ketorolac in comparison to other commercially available ketorolac products. The aqueous ophthalmic solution of the present invention comprises mixtures of carboxymethyl cellulose, e.g. sodium carboxymethyl cellulose, and has a pH within the range of from about 6.8 to about 7.4, which is comfortable when topically applied to the eye of a patient, wherein the concentration of carboxymethyl cellulose and, preferably, the pH, is selected to provide an increased absorption of ketorolac in the eye of a patient as compared to comparative ketorolac solutions. That is, the absorption of ketorolac may be 130% or greater than the absorption of a comparative aqueous ketorolac ophthalmic solution having the same or higher concentration of ketorolac.

More preferably, the aqueous ophthalmic solution of this invention has a pH within the range of from about 6.8 to about 7.4, particularly 6.8.

More preferably, the aqueous ophthalmic solution of the present invention has a concentration of sodium carboxymethyl cellulose of from about 0.2 to about 2 percent, by weight, even more preferably from about 0.5 to about 1.5 percent, by weight, and most preferably about 0.5% w/v with a ratio of medium and high viscosity sodium carboxymethyl cellulose of 0.325% w/v to 0.175% w/v.

More preferably, the aqueous ophthalmic solution of the invention comprises an effective amount of ketorolac of from 0.40 to 0.50 percent, by weight, and more preferably 0.45 percent, by weight.

More preferably, the aqueous ophthalmic solution of the invention has a viscosity of from 5 to 50 cps, preferably from 10 to 30 cps.

It has been surprisingly discovered that optimizing the concentration of ketorolac tromethamine reduces the occurrence of adverse events while maintaining clinical efficacy. Additionally, it has been discovered that the optimized concentration of ketorolac tromethamine in combination with mixtures of carboxymethyl cellulose offers surprising and clear benefits in terms of formulation in that no preservative, chelating agent, or surfactant are required for formulation. Thus, finding a way to increase the absorption of ketorolac benefits the patient who can use a solution having an optimized concentration of ketorolac and obtain similar results in terms of efficiency as compared to a ketorolac solution having a higher concentration of ketorolac.

Thus, this invention relates to an aqueous topical ophthalmic composition comprising 0.40 to 0.50 percent by weight and most preferably 0.45% ketorolac tromethamine by weight/volume of total composition. The present invention also contains from 0.2 to 2 percent by weight, more preferably from 0.5 to 1.5 percent by weight and most preferably about 0.5% w/v percent of medium and high molecular weight sodium carboxymethyl cellulose. Another aspect of this invention relates to a method of treating or preventing ocular pain in a person comprising topically administering to said patient a sterile composition comprising from 0.40% to 0.50% by weight, or 0.45% w/v ketorolac tromethamine in combination with from 0.2 to 2 percent, by weight, preferably from 0.5 to 1.5 percent by weight, and most preferably 0.5% percent by weight/volume, of mixtures of sodium carboxymethyl cellulose.

While not intending to limit the scope of this invention in any way, of particular interest is the use of aqueous topical ophthalmic compositions of 0.45% (w/v) ketorolac tromethamine for the treatment of ocular pain, particularly for the treatment of ocular pain in postoperative photorefractive keratectomy (PRK) surgery patients which also improves healing. It has been surprisingly discovered that the lower concentration of ketorolac as compared to the Acular® product, discussed herein, reduces the incidence of adverse events and enhances comfort, while maintaining clinical efficacy. Two drops (0.1 mL) of 0.5% ketorolac tromethamine ophthalmic solution instilled into the eyes of patients 12 hours and 1 hour prior to cataract extraction achieved measurable levels in 8 of 9 patients' eyes (mean ketorolac concentration 95 ng/mL aqueous humor, range 40 to 170 ng/mL). Ocular administration of ketorolac tromethamine reduces prostaglandin E₂ (PGE₂) levels in aqueous humor. The mean concentration of PGE₂ was 80 pg/mL in the aqueous humor of eyes receiving vehicle and 28 pg/mL in the eyes receiving 0.5% ketorolac tromethamine ophthalmic solution.

Ocular administration of the 0.45% w/v ketorolac tromethamine ophthalmic solution of the present invention increases relative bioavailability of ketorolac in the aqueous humor of rabbits to greater than 200% and in the iris-ciliary body to nearly 300% compared with 0.5% ketorolac tromethamine ophthalmic solution. This enhanced ketorolac bioavailability allows for a reduction in dosing frequency from QID with 0.5% ketorolac tromethamine ophthalmic solution to BID with 0.45% ketorolac solution. Preclinical data indicate systemic ketorolac exposure levels achieved following ocular administration of 0.45% ketorolac solution are comparable to levels achieved with 0.5% ketorolac tromethamine ophthalmic solution.

Ocular administration of 0.45% w/v ketorolac tromethamine ophthalmic solution of the present invention has also been shown to increase visual acuity of the user from I line of improvement of visual acuity and up to greater than 3 lines of acuity.

### BRIEF DESCRIPTION OF THE DRAWINGS

FIG. 1 shows that use of the present invention improves visual acuity of patients in comparison to the vehicle.

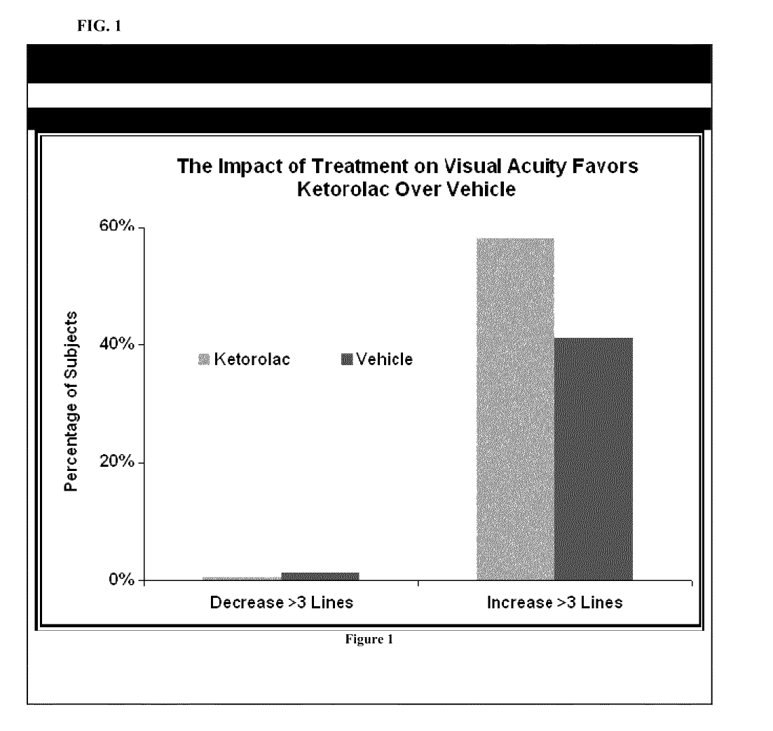

FIG. 2 shows the ocular pharmacokinetics of the aqueous humor of the increased and prolonged ketorolac exposure of 0.45% ketorolac in comparison to 0.40% keterolac (ACULAR LS).

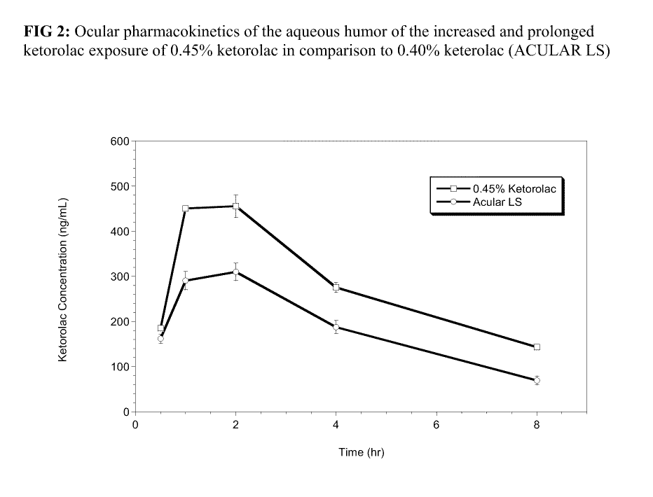

FIG. 3 shows the ocular pharmokinetics in the iris-ciliary body of 0.45% keterolac compared to 0.40% keterolac (ACULAR LS).

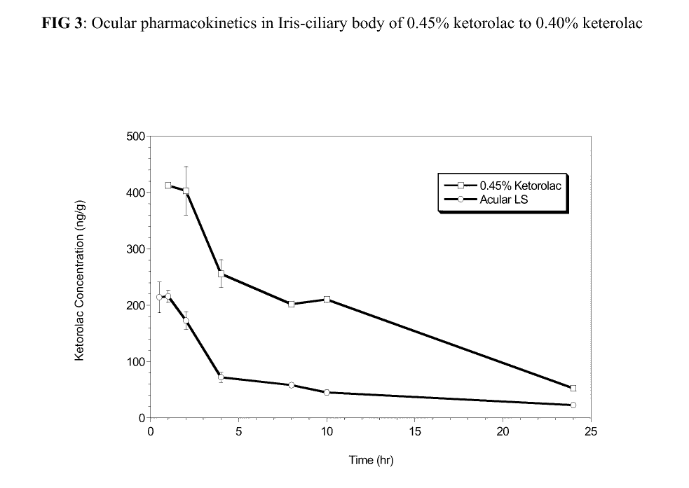

FIG. 4 shows a multiple dose simulation in the iris ciliary body of 0.45% keterolac BID vs. ACULAR LS.

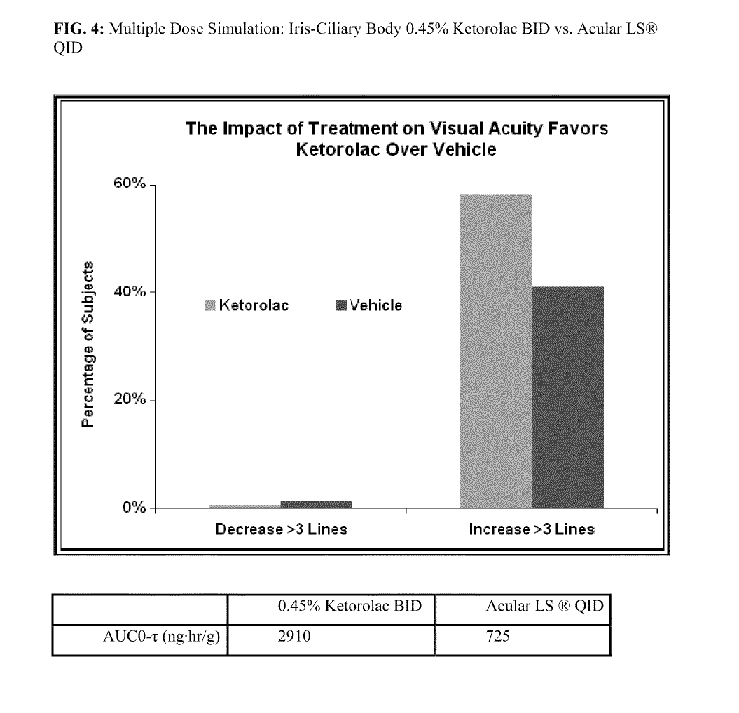

### DETAILED DESCRIPTION OF THE INVENTION

During the reformulation of Allergan's marketed Acular LS® product (0.40% w/v ketorolac), it was surprisingly found that a test formulation containing 0.45% ketorolac tromethamine and mixtures of sodium carboxymethyl cellulose (NaCMC) exhibited significantly better ocular absorption in rabbits than did the currently marketed product, i.e. Acular LS®. Since the viscosities of the two test solutions were virtually identical, the mechanism for achieving increased ocular penetration compared to the control formulation cannot be accounted for by the viscosity of the test solutions. In fact, a comparison of two identical carboxymethyl cellulose-containing solutions which differ only in having viscosity of 11 and 22 cps shows similar absorption of ketorolac into the aqueous humor. While not wishing to be bound by any theory, it is believed that there is a functional relationship between sodium carboxymethyl cellulose and the ketorolac or some component of the ocular surface that facilitates absorption of ketorolac in users.

All of the aqueous topical ophthalmic solutions of this invention are contemplated for use in treating or preventing ocular pain. Preferably, all of the solutions of this invention are contemplated for use when said ocular pain is a result of photorefractive keratectomy surgery (PRK).

One important aspect of this invention is that the solutions of the present invention have a concentration of ketorolac tromethamine which is optimized to reduce side effects and patient compliance, while maintaining clinical efficacy in treating ocular pain. As such, the concentration of ketorolac tromethamine in compositions related to this invention is preferably from 0.35% to 0.50% by weight or w/v, most preferably the concentration of ketorolac tromethamine in the aqueous topical ophthalmic solution of this invention is 0.45% ketorolac tromethamine, by weight.

Carboxymethyl cellulose (CMC) is a carboxymethyl derivative of cellulose formed by the reaction of cellulose with alkali and chloroacetic acid. As a result of said reaction, carboxymethyl groups are bound to some of the hydroxyl groups of the glucopyranose units that make up the backbone of cellulose. The degree of substitution of carboxymethyl varies from about 0.6 to 0.95 per glucopyranose unit. CMC is used in aqueous solutions usually as the sodium salt to increase viscosity.

Carboxymethyl cellulose is available in various molecular weights. Low molecular weight carboxymethyl cellulose has a Mw of about 90,000 and a 2% solution thereof will have a viscosity of about 1.1 cP at 25° C. Medium weight carboxymethyl cellulose has a Mw of about 250,000. High molecular weight carboxymethyl cellulose has a Mw of about 700,000 and a 2% solution will have a viscosity of about 12 cP at 25° C.

For the purpose of the present invention, it is necessary to use a mixture of medium and high molecular weight sodium carboxymethyl cellulose. For example, from 25/75 to 75/25 carboxymethyl cellulose, preferably from 30/70 to 70/30 and most preferably about 35/65 medium/high molecular weight sodium carboxymethyl cellulose.

The fact that the concentration of ketorolac tromethamine in compositions of the present invention achieve greater ketorolac absorption into the aqueous humor of the eye and includes carboxymethyl cellulose in comparison to prior art inventions, allows the solutions of the present invention to be prepared with no preservative, surfactant and chelating agent. This is a significant advantage over prior art ketorolac formulations as preservatives, surfactants and chelating agents can cause irritation to the eye of the user resulting in less patient compliance and less effectiveness of prior art ketorolac formulations.

The term preservative has the meaning commonly understood in the ophthalmic art. Preservatives are used to prevent bacterial contamination in multiple-use ophthalmic preparations, and, while not intending to be limiting, examples include benzalkonium chloride, stabilized oxychloro complexes (otherwise known as Purite®), phenylmercuric acetate, chlorobutanol, benzyl alcohol, parabens, and thimerosal. Preferably, the ketorolac solution of the present invention is preservative free.

The term surfactant used in this invention has the meaning commonly understood in the art. Surfactants are compounds used to help solubilize the therapeutically active agent or other insoluble components of the composition. Anionic, cationic, amphoteric, zwitterionic, and nonionic surfactants may all be used in this invention. If a surfactant is included in the solutions of this invention, preferably, a nonionic surfactant is used. While not intending to limit the scope of the invention, some examples of useful nonionic surfactants are polysorbates, poloxamers, alcohol ethoxylates, ethylene glycol-propylene glycol block copolymers, fatty acid amides, and alkylphenol ethoxylates, and phospholipids. Most preferably, the surfactant is an octylphenol ethoxylate with an average of 40 ethoxylate groups. This type of surfactant, also known as octoxynol-40 or Igepal CA-897®, can be purchased under the Igepal CA-897® trade name from Rhone-Poulenc. Preferably, the ketorolac solution of the present invention is surfactant free.

The term chelating agent refers to a compound that is capable of complexing a metal, as understood by those of ordinary skill in the chemical art. Chelating agents are used in ophthalmic compositions to enhance preservative effectiveness. While not intending to be limiting, some useful chelating agents for the purposes of this invention are edetate salts like edetate disodium, edetate calcium disodium, edetate sodium, edetate trisodium, and edetate dipotassium. Preferably, the ketorolac solution of the present invention is chelator free.

In addition to surfactants, preservatives, and chelating agents, tonicity agents and other excipients are often used in ophthalmic compositions. Tonicity agents are often used in ophthalmic compositions to adjust the concentration of dissolved material to the desired isotonic range. Tonicity agents are known to those skilled in the ophthalmic art, and, while not intending to be limiting, some examples include glycerin, mannitol, sorbitol, sodium chloride, and other electrolytes. Preferably, the tonicity agent is sodium chloride.

One preferred embodiment of this invention relates to an aqueous topical ophthalmic composition comprising 0.4%-0.5% ketorolac tromethamine, from 0.2 to 2.0%, by weight, sodium carboxymethyl cellulose.

The most preferred embodiment of this invention relates to an aqueous topical ophthalmic composition consisting of 0.45% (w/v) of ketorolac tromethamine, 0.5% w/v of carboxymethyl cellulose sodium, e.g. a mixture of medium and high viscosity sodium carboxymethyl cellulose, sodium chloride, sodium citrate dehydrate, sodium hydroxide, hydrochloric acid and purified water.

### Example 1

Unless otherwise specified, all steps in this procedure were carried out at room temperature. The following procedure was followed in accordance with the amounts listed in Table 1 below. Purified water was charged into the main batch vessel. Mixing was initiated to produce a vortex sufficient to disperse and/or dissolve all product ingredients without excessive aeration or foam formation. The following components were added directly into the vortex in order, allowing each to dissolve before adding the next: sodium chloride, calcium chloride, dihydrate magnesium chloride, hexahydrate, boric acid, sodium borate, sodium carboxymethyl cellulose as a percent aqueous solution comprising a mixture of 65% medium molecular weight and 35% high molecular weight carboxymethyl cellulose. The solution was mixed for no longer than 15 minutes. A specified amount of 1N sodium hydroxide was then added. The pH was checked and, if needed, was adjusted to 7.3 with 1N sodium hydroxide or 1N hydrochloric acid. Ketorolac tromethamine was then added based on “as is” assay and mixed until completely dissolved based on visual inspection. When dissolved, the solution pH was again checked and if needed adjusted to pH 7.3-7.5 (final target pH is 7.4) with 1N sodium hydroxide or 1N hydrochloric acid. Purified water was then added to bring the bulk solution to final volume and allowed to mix for at least 15 minutes to ensure uniformity. The solution was then sterile filtered for use.

TABLE 1
0.4% Ketorolac Tromethamine Ophthalmic Solution of the Invention

| | |
|---|---|
| Ketorolac Tromethamine | 0.4% |
| CMC, Med Visc. | .65% |
| CMC Low Visc. | .35% |
| Potassium chloride | 0.14% |
| Calcium chloride, dihydrate | 0.060% |
| Magnesium chloride, hexahydrate | 0.060% |
| Boric acid | .060% |
| Sodium borate | .1225% |

### Example 2

Unless otherwise specified, all steps in this procedure were carried out at room temperature. The following procedure was followed in accordance with the amounts listed in Table 2 below. Purified water at 90% of batch size was charged into the main batch vessel. Mixing was initiated to produce a vortex sufficient to disperse and/or dissolve all product ingredients without excessive aeration or foam formation. The following components were added directly into the vortex in order, allowing each to dissolve before adding the next: sodium chloride, edetate disodium, octoxynol-40 (as a 70% stock solution) and benzalkonium chloride (as a 10% stock solution). The amount of benzalkonium chloride added took into account the assay of the stock solution used. The solution was mixed for no longer than 15 minutes. A specified amount of 1N sodium hydroxide, 1.85 mL per liter of final bulk product, was then added. The pH was checked and if needed was adjusted to 10.7-11.0 with 1N sodium hydroxide or 1N hydrochloric acid. Ketorolac tromethamine was then added based on “as is” assay and mixed until completely dissolved based on visual inspection. When dissolved, the solution pH was again checked and if needed adjusted to pH 7.3-7.5 (final target pH is 7.4) with 1N sodium hydroxide or 1N hydrochloric acid. Purified water was then added to bring the bulk solution to final volume and allowed to mix for at least 15 minutes to ensure uniformity. The solution was then sterile filtered for use.

TABLE 2
0.4% Ketorolac Tromethamine Ophthalmic Solution (Comparative)

| | |
|---|---|
| Ketorolac Tromethamine | 0.4% |
| Edetate Disodium | 0.015% |
| NaCl | 0.79% |
| Benzalkonium Chloride | 0.006% |
| Octoxynol-40 | 0.003% |
| Ph | 7.4 |

### Example 3

This example was prepared according to the procedure of Example 1, except that hydroxypropyl cellulose was used in place of the sodium carboxymethyl cellulose in an amount sufficient to provide a viscosity equivalent to the viscosity of the composition of Example 1.

### Example 4

The following composition was manufactured on a volume basis at ambient temperate from two principal parts. Each part is manufactured separately and then combined under controlled sequences to form a sterile bulk product: the first part (Part 1) involves the dissolution of carboxymethyl cellulose sodium in water followed by bulk heat sterilization, and the second part (Part 2) involves dissolution of ketorolac tromethamine and salts in water sterile filtration through a 0.2 micron membrane into a sterile pressure vessel. The sterile bulk solution is then clarity filtered through a 20 micron polypropylene membrane filter into the filling machine reservoir.

The sterile post-clarity filtered solution is then filled by a UD filling machine via blow-fill-seal process into UD vials using virgin LDPE resin without colorant. The filling is done in an ISO Class 5 environment. The nominal fill is 0.4 mL into 0.9 mL capacity vials.

TABLE 3
0.45 % w/v Ketorolac Tromethamine Ophthalmic Solution

| | | Concentration |
|---|---|---|
| Ingredient | Function | (% w/v) |
| Ketorolac tromethamine | Active | 0.45% |
| Carboxymethycellulose | Thickening Agent | 0.325% |
| Sodium (Med. Viscosity) | | |
| Carboxymethycellulose | Thickening Agent | 0.175% |
| Sodium (High Viscosity) | | |
| NaCl | Tonicity Agent | 0.7% |
| Sodium Citrate Dihydrate | Buffer | 0.2% |
| Sodium Hydroxide (1N) | pH adjustment | Adjust to pH 6.8 |
| Hydrochloric Acid (1N) | pH adjustment | Adjust to pH 6.8 |
| Purified Water | Vehicle | Q.S. |

### Example 5

Comparison of Aqueous Humor Ketorolac Pharmacokinetics Following a Single Ocular Instillation of 0.45% Ketorolac Tromethamine Formulations with Varying pH to Acular LS® in New Zealand White Rabbits.

Study Objectives:

1) To compare aqueous humor ketorolac pharmacokinetics following a single ocular instillation of 0.45% ketorolac tromethamine formulations with varying pH and Acular LS® to New Zealand White rabbits;
2) This Example was designed to determine whether decreasing the pH of the composition would increase the absorption of ketorolac into the eye; and,
3) In addition, one arm of this trial was designed to test the effect of decreasing viscosity of the composition from 22 cps to 11 cps.

The specifics of this study are as follows:

Rabbit Aqueous Humor Ketorolac Concentrations following Administration of Three 0.45% Ketorolac Tromethamine Formulations and Acular LS

TABLE 4

| | |
|---|---|
| Treatment Groups | 0.45% Ketorolac Tromethamine 22 cps pH = 7.4 |
| | 0.45% Ketorolac Tromethamine 22 cps pH = 7.2 |
| | 0.45% Ketorolac Tromethamine 22 cps pH = 7.0 |
| | 0.45% Ketorolac Tromethamine 11 cps pH = 7.0 |
| | 0.45% Ketorolac Tromethamine 22 cps pH = 6.8 |
| | Acular LS pH = 7.4 |
| Dosing Route: | Topical ocular |
| Animal Gender: | NZW Rabbits/Female |
| Dosing Regimen | Single dose, bilateral |
| Timepoints: | 1, 2 and 4 hrs post-dose |
| # Rabbits: | 3 rabbits/timepoint + 1 rabbit blank |
| | Total = 39 rabbits |
| Tissues/Matrices: | Aqueous Humor |
| Bioanalysis: | LC-MS/MS |
| Data analysis: | AUC₀₋ₜ, Cₘₐₓ |

The results of the study are reported in Table 5, below.

TABLE 5
PK Parameters

| | AUC₀₋₄ ± SE | Cₘₐₓ ± SD | |
|---|---|---|---|
| Formulation | (ng · h/ml) | (ng/ml) | Relative % F* |
| 0.45% CMC 22 | 627 ± 51 | 265 ± 71 | 135 |
| cps | | | |
| pH 7.4 w.o | | | |
| “outlier” | | | |
| 045% CMC 22 | 713 ± 96 | 322 ± 153 | 153 |
| cps | | | |
| pH 7.4 | | | |
| 045% CMC 22 | 620 ± 50 | 240 ± 84 | 133 |
| cps | | | |
| pH 7.2 | | | |
| 045% CMC 22 | 658 ± 73 | 268 ± 125 | 142 |
| cps | | | |
| pH 7.0 | | | |
| 045% CMC 22 | 939 ± 163 | 389 ± 258 | 202 |
| cps | | | |
| pH 6.8 | | | |
| 045% CMC 11 | 649 ± 74 | 347 ± 218 | 139 |
| cps | | | |
| pH 7.0 | | | |
| Acular LS ® | 465 ± 65 | 211 ± 106 | 100 |

### Summary of the Results

The sodium carboxymethyl cellulose-containing formulations perform better than Acular LS® with a relative bioavailability ranging from 133% (0.45% Keto 22 cps pH 7.2) to 202% (0.45% Keto 22 cps pH 6.8). However, there is not a clear pH effect-because the 0.45% Keto 22 cps pH 7.4 has a relative bioavailability of 153%, although one anomalous result maybe driving this observation. Nevertheless, the solution having a pH of 6.8 shows the best bioavailability.

### Example 6

A multicenter, randomized, double-masked, parallel-group study is carried out using the 0.4% ketorolac tromethamine formulations of Examples 2 and 3. The study subjects consisted of 157 patients (78-79/group) undergoing unilateral PRK surgery. The key inclusion criteria for the study is that each subject a) is a candidate for unilateral photorefractive keratectomy surgery (PRK) within 7 days after the initial visit, b) have best-corrected ETDRS visual acuity of 20/100 or better, and c) is capable of wearing a soft bandage contact lens. Key exclusion criteria are a history of refractive ocular surgery and sensitivity to study medication or its vehicle, Tylenol #3°, or Ocuflox®. The patient demographics are shown in Table 6. A total of 157 patients are enrolled with an age range of 20-66 years. There are no significant demographic differences between treatment groups.

TABLE 6
Patient Demographics

| | n | | % |
|---|---|---|---|
| Gender | | | |
| Female | 91 | | 58 |
| Male | 66 | | 42 |
| Age, mean ± SD | 39 | ± 10 | |
| Race | | | |
| Caucasian | 148 | | 94 |
| Black | 5 | | 3 |
| Hispanic | 2 | | 1 |
| Asian | 1 | | 1 |
| Other | 1 | | 1 |

Each subject receives the Ocuflox® 5 min prior to study medication. The study subjects then receive ketorolac tromethamine 0.4% ophthalmic solution of Example 2 or Example 3, 1 drop QID for up to 4 days. Then all subjects are then instructed to take Tylenol #3® as needed for intolerable pain (escape medication). Patients use electronic diaries with date and time stamp to record the ocular pain they experience as one of the following: no pain, mild, moderate, severe, intolerable.

The pain intensity is less for the subjects who receive the solution of Example 2 during the first 12 hours post-PRK compared to those who receive the solution of Example 3. In particular, during the first 12 hours post-PRK, the group that received the solution of Example 2 had fewer patients with severe or intolerable pain compared with the group that received the solution of Example 3. In particular, the median pain intensity reported by the group which received the solution of Example 2 was 1 grade less than with the group which received the solution of Example 3 (moderate vs. severe on a 5-point scale of 0=no pain to 4=intolerable pain). Additionally, pain intensity is also less for the group which received the solution of Example 2 compared with the group which received the solution of Example 3.

This clinical study shows that the solution of invention provides a greater degree of absorption of ketorolac as compared to the solution without sodium carboxymethyl cellulose despite the fact that the solutions have the same concentration of ketorolac and are at the same viscosity.

In summary, the 0.4% ketorolac formulation is clinically effective in treating post PRK ocular pain. In patients treated with 0.4 ketorolac tromethamine—the patients treated with the solution comprising sodium carboxymethyl cellulose experienced significantly greater and faster pain relief, and used less escape medication compared to the patients treated with the solution comprising hydroxypropylcellulose.

### Example 7

### Rabbit Ocular Pharmacokinetic Evaluation of Ketorolac Tromethamine 0.45% NZW Rabbits/Female

| | |
|---|---|
| Dosing Regimen: | Single ocular dose, bilateral |
| Timepoints: | 0.5, 1, 2, 4, 8, 10 and 24 hrs post-dose |
| Tissues/Matrices: | Aqueous Humor and Iris-ciliary body |
| Bioanalysis: | LC-MS/MS |
| Data Analysis: | Pharmacokinetic analyses and simulation |

Table 7: Ocular Pharmacokinetics, Aqueous Humor Relative bioavailability based on AUC0-t comparison of 0.45% Ketorolac to 0.45% Ketorolac Acular LS®

| | 0.45% Ketorolac | Acular LS ® |
|---|---|---|
| Cmax (ng/mL) | 456 | 310 |
| AUC0-t (ng · h/mL) | 2230 | 1467 |
| % Relative Bioavailability | 178 | 100 |

Table 8: Ocular Pharmacokinetics: Iris Ciliary Body, Relative bioavailability based on dose normalized AUC comparison to Acular LS®, Increased and Prolonged Ketorolac Exposure

| | 0.45% Ketorolac | Acular LS ® |
|---|---|---|
| Cmax (ng/g) | 429 | 216 |
| AUC0-∞ (ng · h/g) | 5090 | 1860 |
| % Relative Bioavailability | 285 | 100 |

### Conclusions

1) Increase in relative bioavailability of 0.45% ketorolac as compared to Acular LS®;
2) Increased ketorolac concentrations are maintained longer post-dose; and,
3) Together these data support a reduction in dosing frequency from 4×/day to 2×/day.

TABLE 9
Safety and Tolerability Results of 0.45% ketorolac v. ACULAR LS

| Variable | Ketorolac 0.45% | ACULAR LS 0.40% |
|---|---|---|
| Ocular AEs-Irritation | 10.0% (2/20) | 15.4% (6/39) |
| Symptoms-Burning/stinging | 10.0% (2/20) | 12.8% (5/39) |
| (≧ 1 grade increase) | | |
| Bulbar hyperemia (≧ trace) | 10.0% (2/20) | 23.1% (9/39) |
| Ocular comfort (≧ comfortable) | 90-100% | 84-100% |

### Conclusion:

Acular 0.45% is safe and well-tolerated when given 5 times over a half-day and compares very favorably to ACULAR LS.

### Example 8

### Visual Acuity in Operative Eye

This summary of clinical safety (SCS) is based on 2 completed phase 3 studies of identical design and on one completed phase 1 study. All 3 studies support the safe use of a new formulation of ketorolac tromethamine ophthalmic solution 0.45% (henceforth referred to as ketorolac 0.45%), an unpreserved formulation of ketorolac tromethamine ophthalmic solution containing a mixture of medium and high-molecular carboxymethyl cellulose (CMC). The formulation was used in the phase 3 studies, which investigated the safety of ketorolac 0.45% in cataract surgery patients. The phase 1 study investigated the safety of ketorolac 0.45% in healthy adult volunteers.

The current labeling for approved formulations of ketorolac tromethamine ophthalmic solution such as ACULAR LS® (0.40% w/v ketorolac) lists from 20% to 40% transient stinging and burning on instillation. The 0.45% formulation used in the phase 3 studies that form the basis of this application was developed, among other reasons, to improve comfort when administered in the eye, primarily by the addition of medium and high molecular weight carboxymethyl cellulose and the removal of octoxynol and benzalkonium chloride (BAK).

Studies evaluated efficacy and safety of ketorolac 0.45% compared with vehicle (same composition without the active) for the treatment of anterior chamber inflammation, ocular pain, and inhibition of surgically induced miosis following cataract extraction with posterior chamber intraocular lens (IOL) implantation. Both studies demonstrated that ketorolac 0.45% was safe and well tolerated. No new or unexpected safety findings were observed in either study. In addition, ketorolac 0.45% was found to be very well tolerated with a very low incidence of burning and stinging.

Another study assessed the safety and tolerability of ketorolac tromethamine ophthalmic solutions 0.35% and 0.45% compared with ACULAR LS® 0.4% in healthy adult subjects. ACULAR LS® 0.4% was chosen for comparison as it was known to be a better tolerated formulation than original ACULAR® (0.50% w/v ketorolac) due to the lower concentration of ketorolac and removal of BAK and octoxynol. Compared with ACULAR LS® 0.4%, ketorolac 0.45% had a consistently lower incidence of ocular symptoms such as burning/stinging and ocular discomfort when dosed 5 times in 1 day. No new or unexpected safety findings were observed for any of the ketorolac formulations.

The ketorolac 0.45% formulation of the present invention was characterized in ocular and systemic ketorolac rabbit pharmacokinetic and toxicokinetic studies, in addition to 1-day ocular tolerability, 1-month ocular toxicity, and 6-day ocular wound healing studies. From the results of these preclinical studies, 0.45% ketorolac tromethamine administered twice per day was anticipated to deliver ketorolac to ocular tissues that are efficacious but at lower levels than those previously demonstrated to be safe in long term toxicology studies.

The phase 3 studies were of identical design, multi-center, randomized, double-masked, parallel group comparison studies conducted to assess the safety and efficacy of ketorolac 0.45% compared with vehicle. Study patients underwent cataract extraction surgery with posterior chamber IOL implantation. Ketorolac 0.45% or vehicle was self-administered by patients (1 drop twice daily in the operative eye beginning on the day before surgery and continuing on the day of surgery through the first 2 weeks following surgery) and administered by medical personnel (3 drops during the 2 hr prior to surgery and 1 drop after surgery). The safety parameters assessed were adverse events, vital signs, intraocular pressure, visual acuity, biomicroscopy, and opthalmoscopic examinations. The studies consisted of 7 scheduled visits: screening (week −4 to day −2); randomization (day −3 to day −1); cataract surgery day; and postoperative days 1, 3, 7, and 14. Patients receiving ketorolac 0.45% had a lower incidence of ocular adverse events and of adverse events that led to discontinuation than patients receiving vehicle.

A phase 1 study, a single-center, randomized, double-masked, paired-eye, active-controlled study in healthy adult subjects assessing the safety and tolerability of ketorolac tromethamine ophthalmic solutions 0.35% and 0.45% compared with ACULAR LS® 0.4% (5 doses administered on 1 day). Five times in one day, administered by site personnel, subjects received 1 drop of ketorolac 0.45% in study eye/ACULAR LS® in fellow eye or 1 drop of ketorolac 0.35% in study eye/ACULAR LS® in fellow eye. The safety parameters assessed were ocular symptoms, ocular comfort, adverse events, vital signs, intraocular pressure, visual acuity, biomicroscopy, macroscopic bulbar hyperemia, and opthalmoscopic exam. The study consisted of 2 scheduled visits: screening (day −14 to day −1) and dosing day/exit (day 1). Ketorolac 0.45% had a consistently lower incidence of ocular symptoms and signs compared with ACULAR LS® 0.4%.

TABLE 10
Description of Clinical Efficacy and Safety Studies

| Number | | | | | | | | Diagnosis | |
|---|---|---|---|---|---|---|---|---|---|
| of Study | Start date | Design | | | Subjects | | Gender M/F | and | |
| Centers | End date | Control | Active/control, | Study | entered^{a}/ | | Median Age | Inclusion | Safety |
| Locations | Enrollment | type | Route & Regimen | Objective | completed | Duration | (Range) | Criteria | Endpoints |
| 26 | 15 Oct. | Randomized, | ketorolac | Efficacy | 248/201 | 16 days | 107/141 | Planned | Adverse events, |
| United | 2007 | double- | 0.45% | and safety | | | 70 (40- | cataract | vital signs, |
| States | 15 Apr. | masked, | ophthalmic | | | | 89) | extraction | intraocular |
| | 2008 | parallel, | BID | | | | | with | pressure, |
| | | vehicle- | vehicle BID | | | | | posterior | visual acuity, |
| | | controlled | | | | | | chamber | biomicroscopy, |
| | | | | | | | | IOL | fundus |
| | | | | | | | | implant | examinations |
| 22 | 19 Oct. | Randomized, | ketorolac | Efficacy | 263/222 | 16 days | 111/152 | Planned | Adverse events, |
| United | 2007 | double- | 0.45% | and safety | | | 69 (28- | cataract | vital signs, |
| States | 31 Mar. | masked, | ophthalmic | | | | 94) | extraction | intraocular |
| | 2008 | parallel, | BID | | | | | with | pressure, |
| | | vehicle- | vehicle BID | | | | | posterior | visual acuity, |
| | | controlled | | | | | | chamber | biomicroscopy, |
| | | | | | | | | IOL | fundus |
| | | | | | | | | implant | examinations |

Abbreviations: IOL = intraocular lens
^{a}Intent to treat (ITT) population

TABLE 11
Description of Clinical Safety Study

| Number | | | | | | | | Diagnosis | |
|---|---|---|---|---|---|---|---|---|---|
| of Study | Start date | Design | | | Subjects | | Gender M/F | and | |
| Centers | End date | Control | Active/control, | Study | entered/ | | Median Age | Inclusion | Safety |
| Locations | Enrollment | type | Route & Regimen | Objective | completed | Duration | (Range) | Criteria | Endpoints |
| 1 | 03 Jul. | Randomized, | Ketorolac 0.35%, | Safety | 39/39 | 1 day | 14/25 | Healthy | Adverse events, |
| United | 2007 | double- | 0.45%, 5 drops | | | | 24 (19- | adult | ocular symptoms, |
| States | 03 Jul. | masked, | ophthalmic | | | | 63) | volunteers | subject comfort |
| | 2007 | paired-eye, | ACULAR LS ® | | | | | | questionnaires, |
| | | active- | 0.4%, 5 drops | | | | | | vital signs, |
| | | control | | | | | | | intraocular |
| | | | | | | | | | pressure, |
| | | | | | | | | | visual acuity, |
| | | | | | | | | | macroscopic bulbar |
| | | | | | | | | | hyperemia, and |
| | | | | | | | | | biomicroscopy |

In a two phase 3 studies pooled, more than ⅔ of patients in both treatment groups experienced at least 1 line of improvement in visual acuity from baseline to final evaluation. Two patients in each treatment group had a decrease in visual acuity of >3 lines (0.6% [2/320] of ketorolac 0.45% patients, 1.3% [2/158] of vehicle patients).

The proportion of ketorolac 0.45%-treated patients who experienced at least 3 lines of improvement was statistically significantly higher than the proportion of vehicle-treated patients (58.1% [186/320] of ketorolac 0.45% patients, 41.1% [65/158] of vehicle patients, p<0.001). Statistical significance for the same comparison was observed in study −006 (p=0.002) but borderline significant in study −005 (p=0.054).

TABLE 12
Visual Acuity in Operative Eye: Final Evaluation
Compared with Baseline (Safety Population)

| | Study | | Study | | Pooled | |
|---|---|---|---|---|---|---|
| | Ketorolac | | Ketorolac | | Ketorolac | |
| | 0.45% | Vehicle | 0.45% | Vehicle | 0.45% | Vehicle |
| Change (lines)^{a} | (N = 157) | (N = 81) | (N = 173) | (N = 82) | (N = 330) | (N = 163) |
| N | 153 | 78 | 167 | 80 | 320 | 158 |
| ≧+3 | 91 (59.5) | 36 (46.2) | 95 (56.9) | 29 (36.3) | 186 (58.1) | 65 (41.1) |
| <+3 | 62 (40.5) | 42 (53.8) | 72 (43.1) | 51 (63.8) | 134 (41.9) | 93 (58.9) |
| ≧+2 to <+3 | 17 (11.1) | 12 (15.4) | 21 (12.6) | 7 (8.8) | 38 (11.9) | 19 (12.0) |
| ≧+1 to <+2 | 28 (18.3) | 12 (15.4) | 31 (18.6) | 19 (23.8) | 59 (18.4) | 31 (19.6) |
| ≧0 to <+1 | 12 (7.8) | 7 (9.0) | 10 (6.0) | 13 (16.3) | 22 (6.9) | 20 (12.7) |
| ≧−1 to <0 | 4 (2.6) | 4 (5.1) | 5 (3.0) | 6 (7.5) | 9 (2.8) | 10 (6.3) |
| ≧−2 to <−1 | 1 (0.7) | 5 (6.4) | 3 (1.8) | 6 (7.5) | 4 (1.3) | 11 (7.0) |
| >−3 to <−2 | 0 (0.0) | 0 (0.0) | 0 (0.0) | 0 (0.0) | 0 (0.0) | 0 (0.0) |
| ≦−3 | 0 (0.0) | 2 (2.6) | 2 (1.2) | 0 (0.0) | 2 (0.6) | 2 (1.3) |

^{a}positive change represents improvement

In the phase 1 study, most subjects had no change in visual acuity during treatment. Decreases in visual acuity during treatment with respect to baseline were observed for 1 (5.0%) ketorolac 0.45%-treated eye, 1 (5.3%) ketorolac 0.35%-treated eye, and 3 (7.7%) ACULAR LS®-treated eyes.

### Conclusions:

1) The impact of treatment on visual acuity favors ketorolac over vehicle;
2) More than ⅔ of patients in both treatment groups experienced at least 1 line of improvement in visual acuity from baseline to final evaluation;
3) Twice the percentage of patients in the vehicle group experienced a decrease in visual acuity of >3 lines; and,
4) The Acuvail® group experienced over 40% greater incidence of patients experiencing at least 3 lines of improvement—a statistically significant difference.

The present invention is not to be limited in scope by the exemplified embodiments, which are only intended as illustrations of specific aspects of the invention. Various modifications of the invention, in addition to those disclosed herein, will be apparent to those skilled in the art by a careful reading of the specification, including the claims, as originally filed. It is intended that all such modifications will fall within the scope of the appended claims.

What is claimed is:

1. A topical aqueous ophthalmic solution comprising ketorolac, a combination of medium and high molecular weight carboxymethyl cellulose and containing no preservative, wherein the carboxymethyl cellulose is present in the amount of about 0.5 percent by weight, wherein the ketorolac is present in a concentration of about 0.40 to about 0.45 percent by w/v and wherein the solution improves visual acuity of a subject using said solution.

2. The solution of claim 1 wherein the ketorolac is present in the amount of about 0.45 percent by weight.

3. The solution of claim 1 wherein the carboxymethyl cellulose is a combination of medium and high viscosity sodium carboxymethyl cellulose.

4. The solution of claim 1 having a pH within the range of from about 6.8 to about 7.4.

5. The solution of claim 4 having a pH of about 6.8.

6. The solution of claim 2 wherein the solution is surfactant and chelator free.

7. The solution of claim 1 wherein the ketorolac is ketorolac tromethamine.

8. The solution of claim 2 further comprising a mixture of medium and high viscosity sodium carboxymethyl cellulose, sodium chloride, sodium citrate dehydrate, sodium hydroxide, hydrochloric acid and purified water.

9. The solution of claim 7 wherein the ketorolac tromethamine is present as a racemic mixture of R-(+) and S-(−)-ketorolac tromethamine.

10. The solution of claim 9 wherein the ketorolac tromethamine is present in a mixture of crystal forms.

11. The solution of claim 1 wherein the viscosity is from about 10 to about 30 cps.

12. The solution of claim 1 wherein the carboxymethyl cellulose is present in the solution at a ratio of about 0.325% w/v and about 0.175% w/v.

13. A topical aqueous ophthalmic solution consisting of the ingredients listed in Table 3 wherein the solution improves visual acuity of a subject using said solution.

14. A method of improving visual acuity in a subject comprising administration of a therapeutically effective amount of the solution of Table III wherein visual acuity of said subject is improved in comparison to the visual acuity of said subject prior to administering the solution.

15. The method according to claim 14, wherein the visual acuity is improved by at least one line.

16. The method according to claim 15, wherein the visual acuity is improved by at least three lines.

17. The method according to claim 16 wherein the administration of the solution to said subject is twice daily.
